(19) United States
(12) Patent Application Publication
(10) Pub. No.: US 2017/0259072 A1
(43) Pub. Date: Sep. 14, 2017

(54) SYSTEM ARCHITECTURE FOR MEDICAL IMPLANT

(71) Applicant: QUALCOMM Incorporated, San Diego, CA (US)

(72) Inventors: Adam Edward Newham, Poway, CA (US); Rashid Ahmed Akbar Attar, San Diego, CA (US); Anand Palanigounder, San Diego, CA (US)

(21) Appl. No.: 15/454,863

(22) Filed: Mar. 9, 2017

Related U.S. Application Data
(60) Provisional application No. 62/308,121, filed on Mar. 14, 2016.

(51) Int. Cl. A61N 1/372; A61N 1/378; H04W 76/02; A61N 1/36

(52) U.S. Cl. CPC A61N 1/37229; A61N 1/36135; A61N 1/37264; A61N 1/3787; H04W 76/023; H04W 4/008

(57) ABSTRACT

Aspects of the subject matter described in this disclosure can be implemented in an implant device capable of being configured by an external hospital interrogator device when the external hospital interrogator device is authenticated, and capable of communicating data regarding a patient when paired with an external home interrogator device. The implant device includes RF communications circuitry, one or more sensors configured to measure and/or collect data regarding the patient, and a control system. The control system is configured to receive instructions from the external hospital interrogator device for configuring the implant device when the external hospital interrogator device is authenticated, and receive identification data from the external hospital interrogator device for pairing the implant device with the external home interrogator device.

### CROSS-REFERENCE TO RELATED APPLICATIONS

This disclosure claims priority to U.S. Provisional Patent Application No. 62/308,121 (Attorney Docket No. QUALP409P/162113P1), filed Mar. 14, 2016, and entitled “SYSTEM ARCHITECTURE FOR MEDICAL IMPLANT,” which is hereby incorporated by reference in its entirety and for all purposes.

### TECHNICAL FIELD

This disclosure relates generally to implantable medical devices, and more particularly, to a system architecture for securely pairing and configuring implantable medical devices.

### DESCRIPTION OF RELATED TECHNOLOGY

Developments in sensors, electronics, and power source miniaturization have allowed for advancements in medical implant technology. Many implant devices can now be introduced into a patient's body to gather medical data on the patient, monitor the patient's condition, and deliver therapy to the patient. Currently, implant devices are being used in many different parts of the body for various applications, such as orthopaedics, pacemakers, cardiovascular stents, defibrillators, neural prosthetics, neuro stimulation, or drug delivery. The performance, safety, and security of such implant devices can be critical to improving the quality of life of millions of patients. Several challenges can limit the performance and effectiveness of implant devices, including challenges related to power consumption of the implant devices. Additional challenges can limit the safety and security of implant devices, including challenges related to secure communication between implant devices and outside devices.

Power consumption is a big concern in many implantable medical devices, especially battery-powered implantable medical devices. Some implantable medical devices are configured to wirelessly communicate data with a device outside of a patient's body. Various implantable medical devices, such as neuro stimulators, defibrillators, and pacemakers, etc. need to manage power consumption effectively while also reliably communicating data wirelessly.

Providing secure communications between implant devices and outside devices can also present a challenge. Instead of having wireless transmitters and receivers situated outside of a patient's body, the implant devices can be equipped to communicate wirelessly to a device outside of the body. Not only can it be difficult to wirelessly communicate with the implant device subcutaneously, but such communication may be vulnerable to different security threats.

More can be done to develop a system architecture that provides power delivery and power savings as well as secure communications between implant devices and outside devices.

### SUMMARY

The systems, methods, and devices of this disclosure each have several aspects, no single of which is solely responsible for the desirable attributes disclosed herein.

One aspect of the subject matter described in this disclosure can be implemented in an implantable medical device. The implantable medical device includes a radio-frequency (RF) communications circuitry, one or more sensors configured to measure and/or collect physiological data, and a control system coupled to the RF communications circuitry and to the one or more sensors. The control system is configured to: receive instructions from a first external interrogator device for configuring the implantable medical device when the first external interrogator device is authenticated, and receive identification data from the first external interrogator device for pairing the implantable medical device with a second external interrogator device.

In some implementations, the identification data includes a secret key or public/private key pairs provided by the first external interrogator device. In some implementations, the first external interrogator device is provisioned with user credentials for authenticating one or more users to access the first external interrogator device. In some implementations, the control system is configured to cause the RF communications circuitry to transmit the physiological data to the second external interrogator device when the second external interrogator device is paired with the implantable medical device. In some implementations, the second external interrogator device includes a carrier board and a computing device, where the carrier board includes an RF unit for wirelessly communicating with the implantable medical device and the computing device includes a wireless communications component for wirelessly communicating with a wireless communication hub or a cellular device. In some implementations, the implantable medical device further includes a wireless charger and a rechargeable battery coupled to the wireless charger, where the wireless charger is configured to receive signals to wirelessly charge the rechargeable battery in a mid-field frequency range between about 100 MHz and about 5 GHz or in a near-field frequency range. The control system is further configured to: receive a trigger signal from the wireless charger, and pair the implantable medical device with the second external interrogator device upon receipt of the trigger signal. In some implementations, the control system is configured to receive instructions from the first external interrogator device via a local area network or direct physical connection.

Another aspect of the subject matter described in this disclosure can be implemented in an interrogator device. The interrogator device includes a first wireless communications component configured to communicate subcutaneously with an implant device, a second wireless communications component configured to communicate with an electronic device external to the implant device, and a control system. The control system is configured to receive identification data for pairing with the implant device, identify the implant device using the identification data to pair the interrogator device with the implant device, and receive physiological data from the implant device using session keys established using an authenticated key agreement protocol.

In some implementations, the identification data includes a secret key or public/private key pairs provided to the implant device by a remote device. The remote device is provisioned with user credentials for authenticating one or more users to access the remote device. In some implementations, the interrogator device further includes a carrier board and a computing device, where the carrier board includes the first wireless communications component and the computing device includes the second wireless communications component, where the carrier board and the computing device are in communication with each other via a bidirectional communication interface. In some implementations, the first wireless communications component is configured to communicate subcutaneously with the implant device in a Medical Implant Communications Service (MICS) frequency band or in a Bluetooth frequency band, and wherein the second wireless communications component is configured to communicate with the electronic device over one or more of a wide area network, personal area network, local area network, near-field communication (NFC) or any combination thereof. In some implementations, the electronic device is a cellular device or a wireless communications hub device, and the electronic device is configured to transmit the physiological data to a cloud-based database system. In some implementations, the interrogator device further includes a wireless charger coupled to the first wireless communications component, where the wireless charger is configured to wirelessly charge the implant device.

Another aspect of the subject matter described in this disclosure can be implemented in an interrogator device. The interrogator device includes a wireless communications component configured to communicate subcutaneously with an implant device, a memory configured to store user credentials for authenticating one or more users to access the interrogator device, and a control system. The control system is configured to authenticate a user to access the interrogator device using the user credentials, establish secure communication between the interrogator device and the implant device, and transmit, via the wireless communications component, instructions to the implant device to configure one or more operations of the implant device.

In some implementations, the control system is further configured to provide identification data to a remote device and the implant device for pairing the remote device and the implant device, where the identification data includes a secret key or public/private key pairs. In some implementations, the control system is configured to transmit instructions to the implant device via a local area network or direct physical connection. In some implementations, the control system is further configured to determine, after authenticating the user to access the interrogator device, that the user has privileges to be able to access features for transmitting instructions to the implant device. In some implementations, the wireless communications component is further configured to wirelessly charge a battery in the implant device.

Another aspect of the subject matter described in this disclosure can be implemented in a method of operating an implant device. The method includes receiving, via one or more sensors of an implant device, physiological data associated with a treatment profile of a patient, receiving non-physiological data from the implant device or from a device external to the implant device, determining that the non-physiological data, alone or in combination with the physiological data, satisfies a condition, and adjusting, via one or more processors of the implant device, an operation of the implant device in response to determining that the condition has been satisfied.

In some implementations, the non-physiological data includes one or more of environmental data, activity data, user data, system condition data, and contextual data. The non-physiological data includes environmental data received from the device external to the implant device, the environmental data including one or more of ambient pressure, temperature, sound, light, humidity, location, air quality, pollen count, carbon dioxide, and odor. In some implementations, adjusting an operation of the implant device includes adjusting a polling rate of the one or more sensors of the implant device.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

The accompanying drawings, which are incorporated herein and constitute part of this specification, illustrate example embodiments of the claims, and together with the general description given above and the detailed description given below, serve to explain the features of the claims.

FIG. 1 shows a schematic diagram illustrating an example system including a remote device and an implant device inside a patient's body according to some implementations.

FIG. 2 shows a block diagram representation of components of an example implant device according to some implementations.

FIG. 3 shows a block diagram representation of components of an example interrogator device according to some implementations.

FIG. 4 shows a system diagram illustrating communication pathways in an example environment including an implant device, a “hospital” interrogator device, a “home” interrogator device, and a cloud-based database system according to some implementations.

FIG. 5 shows a flow diagram illustrating an example method for establishing secure communication between an implant device and an external home interrogator device according to some implementations.

FIG. 6 shows a flow diagram illustrating an example method for transmitting physiological data from an implant device to an external home interrogator device according to some implementations.

FIG. 7A shows a flow diagram illustrating an example method of operating an implant device according to some implementations.

FIG. 7B shows a flow diagram illustrating an example method of operating an implant device according to some other implementations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Various embodiments will be described in detail with reference to the accompanying drawings. References made to particular examples and implementations are for illustrative purposes, and are not intended to limit the scope of the claims.

The described implementations may be implemented in any device, apparatus, or system. In addition, it is contemplated that some of the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, belts, etc., Bluetooth® devices, personal data assistants (PDAs), wireless electronic mail receivers, hand-held or portable computers, netbooks, notebooks, smartbooks, tablets, printers, copiers, scanners, facsimile devices, global navigation satellite system (GNSS) receivers/navigators, cameras, digital media players (such as MP3 players), camcorders, game consoles, wrist watches, clocks, calculators, television monitors, flat panel displays, implantable medical devices, interrogator medical devices, electronic reading devices (e.g., e-readers), mobile health devices, medical devices, computer monitors, auto displays, cockpit controls and/or displays, steering wheels, camera view displays, electronic photographs, electronic billboards or signs, projectors, architectural structures, microwaves, refrigerators, stereo systems, cassette recorders or players, DVD players, CD players, VCRs, radios, portable memory chips, washers, dryers, washer/dryers, parking meters, etc. By way of example, the described implementations may be implemented in an implant device or implantable medical device. For example, the described implementations may be implemented in a battery-powered implantable medical device, such as a neuro stimulator. Some of the described implementations may be implemented in an interrogator device for communicating with an implant device. Some of the described implementations may be implemented in a system including the interrogator device and the implant device. Nonetheless, the teachings are not intended to be limited to the implementations depicted solely in the Figures, but instead have wide applicability as will be readily apparent to one having ordinary skill in the art.

This disclosure relates generally to systems, methods, and devices for providing secure communication between an implant device and one or more remote devices. In some implementations, the implant device is an implantable medical device and the remote device is one or both of a home interrogator device and a hospital interrogator device. The hospital interrogator device is provisioned with credentials for authenticating one or more users to access the hospital interrogator device. Such users are limited to authorized doctors, authorized healthcare professionals, or other authorized users. The hospital interrogator device is configured to program or otherwise configure the implant device upon authentication. Such configuration with the hospital interrogator device may be limited to communications in a short-range communication protocol and may be limited to a local area network or direct physical connection. The hospital interrogator device is further configured to provide identification data to the implant device and the home interrogator device for pairing the implant device with a home interrogator device. Such identification data can include, for example, a secret key or public/private key pairs. The home interrogator device may be configured to receive data regarding a patient from the implant device but is not configured to program or otherwise configure the implant device. In some implementations, the home interrogator device includes a carrier board with a first wireless communications component for communicating with the implant device, and a computing device with a second wireless communications component for communicating with electronic devices external to the implant device. It will be understood that the hospital interrogator device is not necessarily limited to use in a hospital setting and that the home interrogator device is not necessarily limited to use in a home setting, but may be used in different places and settings. Accordingly, the hospital interrogator device may be referred to as a first interrogator device and the home interrogator device may be referred to as a second interrogator device, or vice versa.

Particular implementations of the subject matter described in this disclosure can be implemented to realize one or more of the following potential advantages. The system architecture, including the hospital/home interrogator device and the implant device, provides security mechanisms for protecting the implant device against security threats. By limiting only the hospital interrogator device to be capable of programming the therapy or treatment of the implant device, the implant device is protected against security threats from other client devices, including the home interrogator device. In addition, the hospital interrogator device is protected against unauthorized access with strong authentication and access control, which limits access or operations based on user roles and privileges. Only authorized hospital or health care professionals may access the hospital interrogator device. Furthermore, by limiting the implant device to be configured or programmed by an interrogator device in a local network, the risk of malicious attack from a third party is reduced unless the local network is accessed. Moreover, the interrogator device separates the carrier board for communicating with the implant device and the computing device for communicating with conventional electronic devices such as smartphones, wireless communication hubs, and cloud-based database systems. As a result, any security compromise of the computing device in communicating with conventional devices does not necessarily compromise the carrier board in communicating with the implant device. Not only does the system architecture of the present disclosure provide secure communications with the implant device, but the system architecture also provides power savings and power management of the implant device. Both the implant device and the interrogator device may be equipped with appropriate RF circuitry for subcutaneously recharging a battery in the implant device. Additionally, by manufacturing components of the implant device at process nodes that do not require protection circuitry, the implant device may be manufactured to eliminate or otherwise reduce quiescent power leakage coming from protection circuitry.

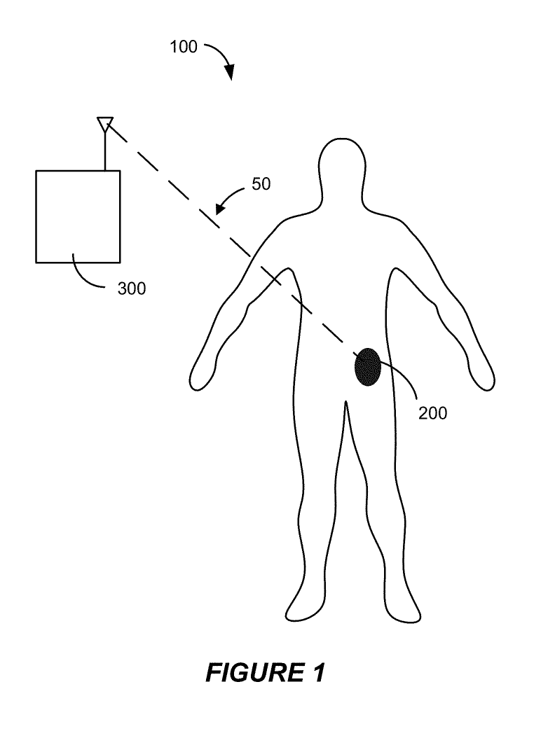

FIG. 1 shows a schematic diagram illustrating an example system including a remote device and an implant device inside a patient's body according to some implementations. The system 100 includes an implant device 200 and a remote device 300. As used herein, the implant device may be referred to as an implantable device or implantable medical device. In some implementations, the implant device 200 may include but is not limited to cardiac pacemakers, implantable cardioverter-defibrillators (ICDs), implantable combination pacemaker-cardioverter defibrillator (PCDs), implantable brain stimulators, implantable gastric system stimulators, implantable nerve or neural stimulators, implantable muscle stimulators, implantable lower colon stimulators, implantable drug dispensers or pumps, implantable cardiac signal loops or other types of recorders or monitors, implantable gene therapy delivery devices, implantable incontinence prevention or monitoring devices, implantable insulin pumps or monitoring devices, and so on. In some implementations, the implant device 200 is battery-powered. The remote device 300 may be configured to wirelessly communicate with the implant device via a secure communication pathway 50. The remote device 300 can be configured to transmit wireless signals via communication pathway 50 and the implant device 200 can be configured to receive the wireless signals. The remote device 300 can be configured to facilitate wireless data transfer between the implant device 200 and the remote device 300. In some implementations, the remote device 300 may include but is not limited to an interrogator device or interrogator medical device, an external medical device, a programming device, a remote telemetry station, a base station for the implant device 200, a physician-activated device, a patient-activated device, a display device, or any other type of device capable of sending and receiving signals to and from the implant device 200.

The scope of the present disclosure is not to be limited to systems including a remote device and an implant device, an implant device only, or a remote device only. Such systems, implant devices, and remote devices are meant to be illustrative and are not intended to limit the scope of the present disclosure or the claims. Various modifications to the implementations described in the present disclosure may be readily apparent to those skilled in the art, and the generic principles defined herein may be applied to other implementations without departing from the spirit or scope of this disclosure. Thus, the present disclosure and the claims are not intended to be limited to the implementations shown herein, but are to be accorded the widest scope consistent with this disclosure, the principles and the novel features disclosed herein.

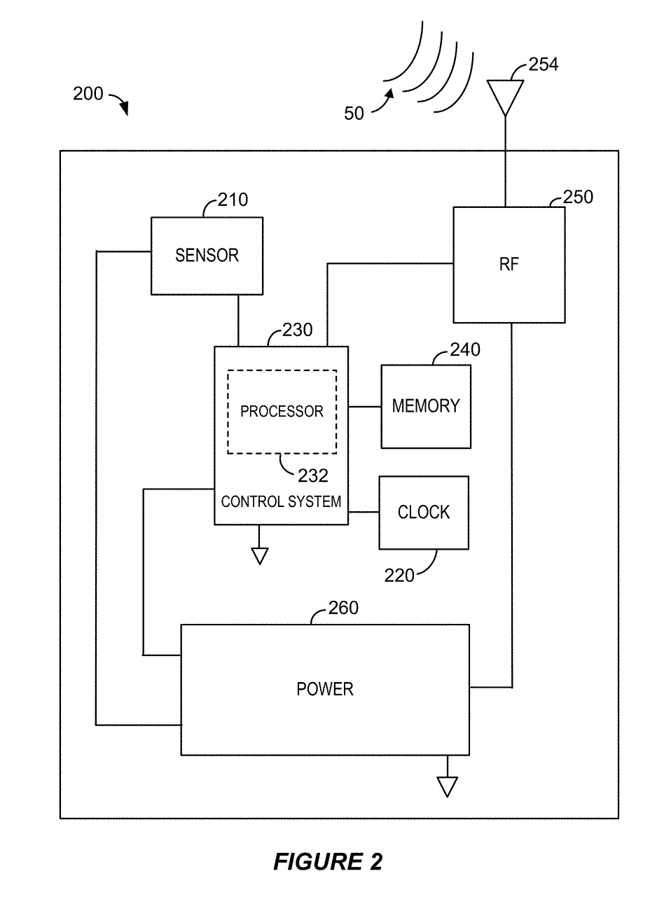

FIG. 2 shows a block diagram representation of components of an example implant device according to some implementations. The implant device 200 may be hermetically sealed and encapsulated in biocompatible material, such as biocompatible glass, ceramic, or titanium. As with other implementations disclosed herein, the number of circuitry elements and types of circuitry elements shown in FIG. 2 are merely by way of example. Other implementations may have more, fewer, or different circuitry elements. In the implementation in FIG. 2, the implant device 200 includes a sensor 210, a clock 220, a control system 230, a memory 240, a wireless communications component 250 coupled to an antenna 254, and a power supply 260.

Some or all of the circuitry elements in FIG. 2 are constructed on one or more semiconductor die. The one or more semiconductor die are arranged to allow for electrically conductive paths between circuitry elements. Each of the semiconductor die may be constructed at a particular fabrication node, where smaller geometry fabrication nodes may increase performance without increasing power consumption. In some implementations, each of the die may contain one or more transistors.

In some implementations, the implant device 200 includes one or more sensors 210. For example, where the implant device 200 is a nerve or neural stimulator, the one or more sensors 210 can be configured to measure the electrical stimulation activity of a nerve. In some implementations, data may be accessed from the one or more sensors 210 by the control system 230 and sent to a remote device, such as a device outside the patient's body.

The implant device 200 can include a clock 220 internal to the implant device 200. The clock 220 may provide timed signals to one or more components of the implant device 200. In some implementations, the clock 220 can include a crystal (e.g., piezoelectric) that oscillates at a particular frequency, such as at 32 KHz. The clock 220 may be fabricated on a die that is separate from a power management integrated circuit (PMIC) die. In some implementations, the clock 220 may be constructed at a fabrication node to allow for direct battery attachment. For example, the clock 220 may be constructed at a fabrication node of 180 nm.

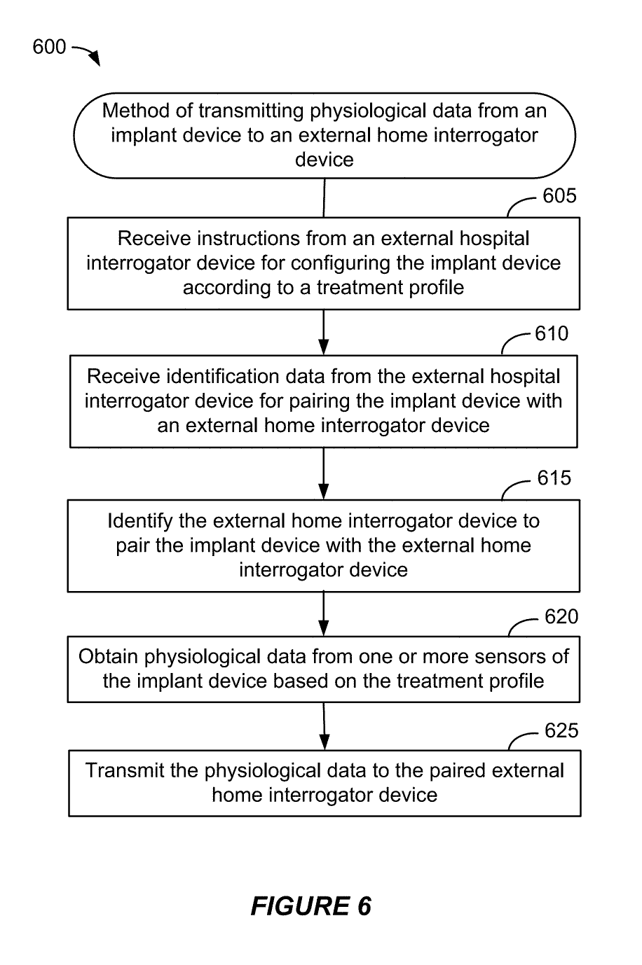

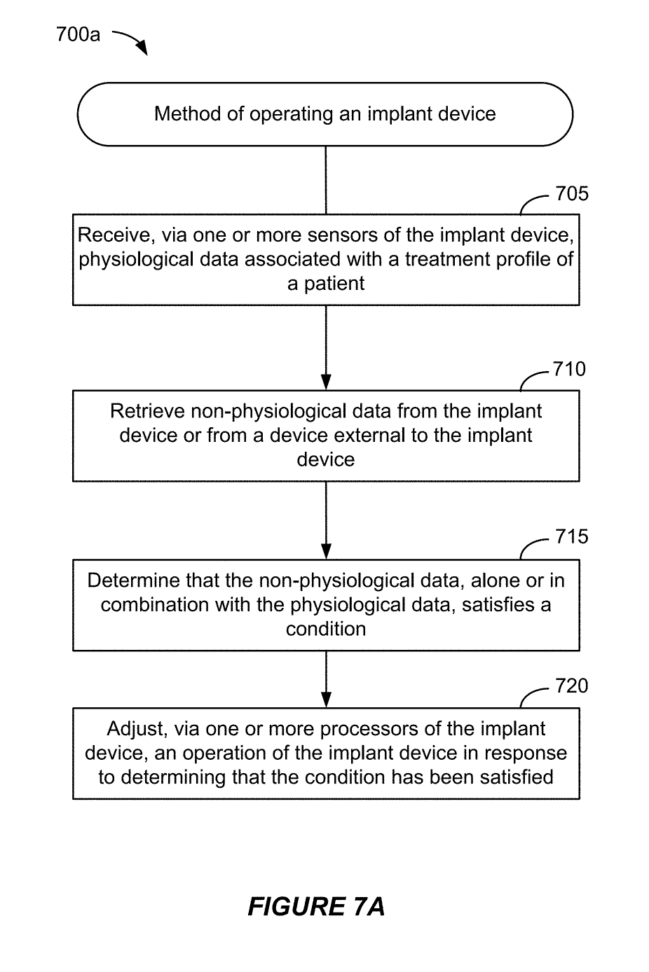

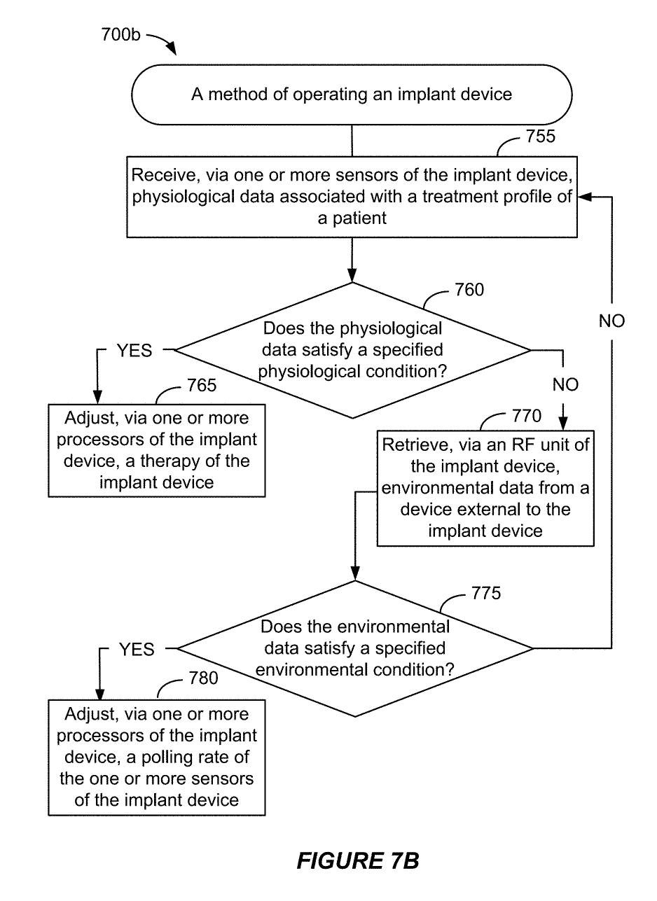

The implant device 200 can include a control system 230. The control system 230 may include at least one of a general purpose single- or multi-chip processor, a digital signal processor (DSP), an application specific integrated circuit (ASIC), a field programmable gate array (FPGA) or other programmable logic device, discrete gate or transistor logic, or discrete hardware components. In some implementations, the control system 230 may include a processor 232. The control system 230 may be capable of performing some or all of the methods described herein. According to some examples, the control system 230 may be capable of performing a method described in a method 600, which is shown in FIG. 6. According to some examples, the control system 230 may be capable of performing a method described in a process 700a or 700b, which is shown in FIGS. 7A and 7B. In some implementations, the control system 230 may be capable of controlling the operations of the implant device 200 by controlling one or more components of the implant device 200. For example, the control system 230 may be capable of controlling the one or more sensors 210. The control system 230 may be capable of controlling the wireless communications component 250.

In implementations where the implant device 200 is a neural stimulator, the control system 230 may control the stimulation pulses for delivery to tissue of the patient. In some implementations, the implant device 200 may be adapted for controlling stimulation pulses in a nerve blocking disease or a nerve stimulation disease. In a nerve blocking disease, an electrical signal may be applied to provide a “blocking” of action potentials traveling along the nerve. In a nerve stimulation disease, an electrical signal may be applied to stimulate a nerve. The electrical power for treatment of nerve blocking diseases may require more power, and in some instances even continuous charging, than treatment of nerve stimulation diseases.

In some implementations, the control system 230 may be capable of controlling the implant device 200 according to instructions (e.g., software) stored on one or more non-transitory computer-readable media. Such non-transitory media may include the memory 240 of the implant device 200. The memory 240 can store processor-executable instructions and/or outputs from the one or more sensors 210. In some implementations, the memory 240 may be a volatile memory, non-volatile memory (e.g., flash memory), or a combination thereof. In some implementations, the memory 240 may include internal memory included in the control system 230, memory external to the control system 230, or a combination thereof. The memory 240 may be coupled to the control system 230. In some implementations, the memory 240 may store information or instructions related to polling of the implant device 200. For example, the memory 240 may store instructions for controlling the polling rate of the one or more sensors 210.

The implant device 200 can include a wireless communications component 250 coupled to an antenna 254. The wireless communications component 250 can include RF circuitry or RF communications circuitry for wirelessly communicating with devices outside a patient's body. The control system 230 may be coupled to the wireless communications component 250 to control the operations of the wireless communications component 250. In some implementations, the wireless communications component 250 may include one or more of a receiver, a transmitter, and a two-way transceiver. The wireless communications component 250 may operate in one or more frequency bands depending on the supported type of communications. The wireless communications component 250 may be configured to receive or transmit signals in a desired frequency band, such as the Medical Implant Communications Service (MICS) band, Medical Electronic Data Service (MEDS) band, near-field communications band, or any other suitable frequency band. In some implementations, the wireless communications component 250 can be configured to receive or transmit at a frequency in the MICS band, where the MICS band is between about 400 MHz and about 405 MHz. In some implementations, the wireless communications component 250 includes an antenna shared with a wireless charger, where the antenna is configured to receive and/or transmit signals in the near-field communications frequency band. The wireless communications component 250 may be constructed on a system-on-chip (SOC) die, where the SOC die can be provided at a fabrication node that supports low dynamic power. For example, the process node for the SOC die can be 28 nm or less.

In some implementations, one or more of the sensor 210, the clock 220, the control system 230, the memory 240, the wireless communications component 250, and any other electronic components of the implant device 200 may be powered by the power supply 260. In some implementations, the power supply 260 may include a rechargeable battery, such as a rechargeable lithium ion battery. In some implementations, the rechargeable battery is recharged by converting a wireless signal into electrical current via a wireless charger. The wireless charger can include an antenna to receive wireless power from a wireless power source. An optimal frequency can be safely and effectively delivered to the antenna, where the frequency can be in the near-field range or in the mid-field range. In the near-field range, the frequency for power transfer can occur at less than about 10 MHz. In the mid-field range, the frequency for power transfer can occur at frequencies ranging from 100 MHz to 5 GHz.

The battery of the power supply 260 may be vulnerable to overcharging or overdischarging conditions. Typically, protection circuitry may be implemented to keep the battery within a safe operating region. Protection circuitry, however, can introduce quiescent power leakage depending on the fabrication nodes of the one or more semiconductor die. Quiescent power comes from the protection circuitry required in order to protect transistors. In some implementations, the power supply 260 is constructed on a PMIC die with switch-mode power supply (SMPS) that allows for direct battery attachment and that can handle voltages up to 5V without requiring protection circuitry. The clock 220 may also be constructed on a semiconductor die at a fabrication node that allows for direct battery attachment without requiring protection circuitry.

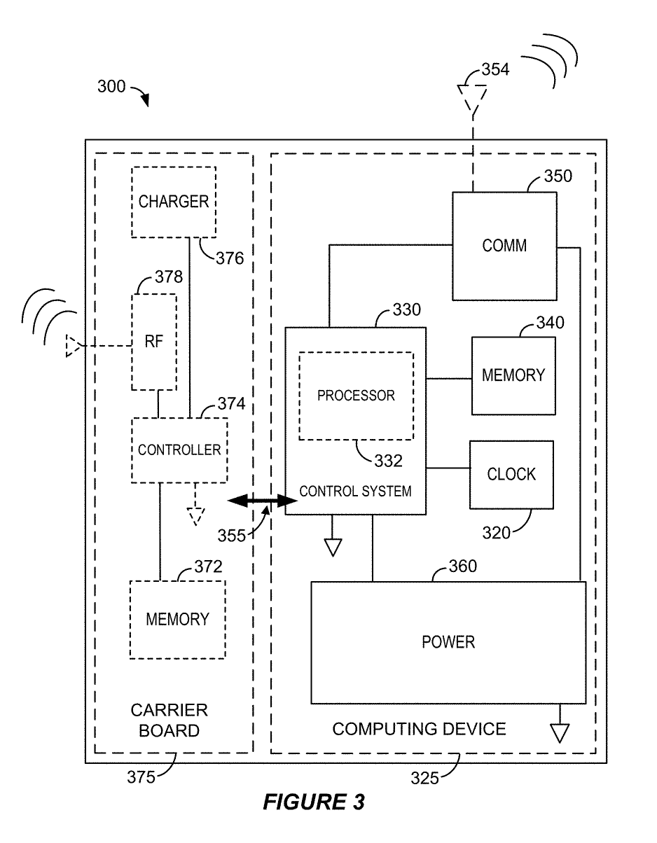

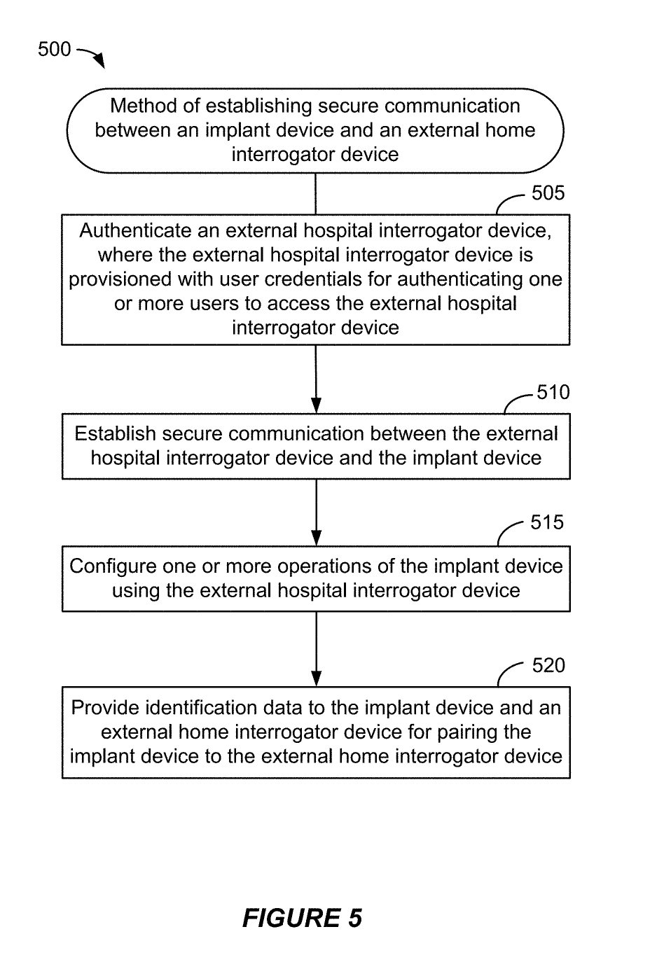

FIG. 3 shows a block diagram representation of components of an example remote device according to some implementations. As with other implementations disclosed herein, the number of circuitry elements and types of circuitry elements shown in FIG. 3 are merely by way of example. Other implementations may have more, fewer, or different elements. In the implementation in FIG. 3, the remote device 300 includes a clock 320, a control system 330, a memory 340, a communications component 350, and a power supply 360. The control system 330 may be capable of performing some or all of the methods described herein. According to some examples, the control system 330 may be capable of performing a method described in a method 500, which is shown in FIG. 5. In some implementations, the remote device 300 includes a computing device 325 and a carrier board 375, where the computing device 325 includes the clock 320, the control system 330 including a processor 332, the memory 340, the wireless communications component 350, and the power supply 360. The carrier board 375 can include a memory 372, a controller 374, a radio-frequency (RF) unit 378, and a charger 376. It is understood that the device 300 is not limited to remote devices, but can include any device capable of sending and receiving signals to and from the implant device 200. Examples of the device 300 can include an interrogator device or interrogator medical device, a programming device, a remote telemetry station, a base station, a physician-activated device, a patient-activated device, or a display device. In some implementations, the device 300 can serve as a base station for receiving data from the implant device 200 and transmitting instructions to the implant device 200.

As shown in FIG. 3, the remote device 300 may include the carrier board 375 and the computing device 325. Separating the carrier board 375 from the computing device 325 can separate the functionality of communicating with the implant device 200, which can involve safety critical features, from the functionality of communicating with non-implanted devices like smartphones, wireless communication hubs, and cloud-based database systems, which do not involve safety critical features. That way, any security compromise of the computing device 325 in communicating with non-implanted devices does not necessarily compromise the carrier board 375 in communicating with the implant device 200. In some implementations, the carrier board 375 of the remote device 300 can include the RF unit 378 (e.g., first wireless communications component) customized to communicate with the implant device 200 in the MICS frequency band, in the Bluetooth frequency band, or with near-field magnetic induction. In some implementations, the computing device 325 of the remote device 300 can include the communications component 350 configured to communicate with non-implanted devices in any appropriate frequency bands, such as Bluetooth or Wi-Fi or cellular. In some implementations, the communications component 350 is configured to communicate across a wired interface, such as Universal Serial Bus (USB) or Ethernet.

In some implementations, a communication link 355 may be provided between the computing device 325 and the carrier board 375 so that a secure connection can be made between the computing device 325 and the carrier board 375. The computing device 325 may be a computer, such as an off-the-shelf computer, single-board computer, or programmable computer system. In some implementations, the communication protocol for the communication link can be Serial Peripheral Interface (SPI), though other suitable communication protocols known in the art, such as Universal Serial Bus (USB), may be applied. The communication link 355 may provide for a robust bidirectional communication interface.

The carrier board 375 may include one or more components for controlling the therapy of the implant device 200 and/or processing the data received from the implant device 200. In some implementations, the carrier board 375 may communicate with the implant device 200 using discrete components rather than application-specific integrated circuits (ASICs). The carrier board 375 may include one or more microprocessors, microcontrollers, field programmable gate arrays, systems-on-a-chip, volatile or non-volatile memory, discrete circuitry, and/or other hardware, software, or firmware.

The carrier board 375 may be equipped to receive data from the implant device 200 and to adjust the treatment of the implant device 200. For example, as the implant device 200 is recording data during a treatment, a doctor or healthcare professional can see the recorded data being downloaded by the remote device 300, and can change the treatment profile by adjusting therapy using the remote device 300. That way, a doctor or healthcare professional can monitor and change treatment using the remote device 300.

The computing device 325 may include one or more microprocessors, microcontrollers, field programmable gate arrays, systems-on-a-chip, volatile or non-volatile memory, discrete circuitry, and/or other hardware, software, or firmware. In some implementations, the computing device 325 can be an off-the-shelf board, such as Raspberry Pi, DragonBoard 410c, 2Net, and the like. Though components of the computing device 325 can include a clock 320, a control system 330, a memory 340, a wired or wireless communications component 350, and a power supply 360, such components may be referred to more generally as components of the remote device 300.

The remote device 300 can include a clock 320, where the clock 320 can serve as a reference clock to accurately represent the time. The clock 320 can be any suitable clock, such as a mechanical clock, quartz clock, pendulum clock, and atomic clock. In some implementations, the clock 320 can be more closely aligned with the reference time and may be continuously powered by the power supply 360.

The remote device 300 can also include a control system 330. The control system 330 may include at least one of a general purpose single- or multi-chip processor, a DSP, an ASIC, an FPGA or other programmable logic device, discrete gate or transistor logic, or discrete hardware components. In some implementations, the control system 330 may include a processor 332. The control system 330 may be capable of performing some of the methods described herein. In some implementations, the control system 330 may be capable of controlling one or more components of the remote device 300. For example, the control system 330 may be capable of executing authentication procedures for accessing some or all of the features of the remote device 300. The control system 330 may be capable of controlling the communications component 350.

In some implementations, the control system 330 may be capable of controlling the remote device 300 according to instructions (e.g., software) stored on one or more non-transitory computer-readable media. Such non-transitory media may include the memory 340 of the remote device 300. The memory 340 can store processor-executable instructions and/or data received from another device. In some implementations, the memory 340 may be a volatile memory, non-volatile memory (e.g., flash memory), or a combination thereof. In some implementations, the memory 340 may include internal memory included in the control system 330, memory external to the control system 330, or a combination thereof. The memory 340 may be coupled to the control system 330. In some implementations, the memory 340 may store information or instructions related to authentication and credentials of the remote device 300.

In some implementations, an antenna 354 may be coupled to the communications component 350 to wirelessly communicate with other devices. The control system 330 may be coupled to the communications component 350 to control the operations of the communications component 350. In some implementations, the communications component 350 may include one or more of a receiver, a transmitter, and a two-way transceiver. While the communications component 350 of the remote device 300 is shown as part of the computing device 325 in FIG. 3, it is understood that the wireless communications component 350 can be part of the carrier board 375 in addition to or in the alternative to the RF unit 378.

In some implementations, the communications component 350 may be configured to communicate over one or more of a wide area network (WAN), personal area network (PAN), local area network (LAN), near-field communication (NFC), USB, Ethernet, or any combination thereof. For example, the communications component 350 can support communication over a personal area network (e.g., Bluetooth). In addition or in the alternative, the communications component 350 can support communication over a wireless local area network (e.g., Wi-Fi). In addition or in the alternative, the communications component 350 can support communication over a wireless wide area network (e.g., LTE). In some implementations, the communications component 350 may be equipped to support communication in the global navigation satellite system (GNSS) network. Examples of GNSS networks include but are not limited to GPS, GLONASS, and BeiDou. The GNSS frequency band can be useful in location-sensing for determining the location of a patient, such as whether the patient is at home, at work, or in the doctor's office. The aforementioned frequency bands are intended to be illustrative and it is to be understood that other frequency bands known in the art can be incorporated without departing from the scope of the present disclosure. As such, the scope of the disclosure is not limited to the description of the above-mentioned frequency bands. In some implementations, the communications component is configured to support communication over a wired interface, such as USB or Ethernet.

In some implementations, the communications component 350 can communicate data received from the implant device 200 to a database system, such as a cloud-based database system, over a wired or wireless interface. In some implementations, the communications component 350 can communicate data received from the implant device 200 to a cellular device, such as a smart phone, mobile phone, smart watch, tablet, PDA, laptop computer, desktop computer, or other device with cellular communication capability, over a wired or wireless interface. For example, the communications component 350 of the computing device 325 may communicate with a cellular device via Bluetooth or Wi-Fi so that data can be sent directly to the cellular device, or the communications component 350 of the computing device 325 may communicate with a cloud-based database system via WWAN. In some implementations, the communications component 350 in the computing device 325 can be differentiated from the RF unit 378 in the carrier board 375, where the communications component 350 is configured to communicate with a database system or a cellular device over a particular communication protocol, and the RF unit 378 is configured to communicate with the implant device 200 over a particular communication protocol. These particular communication protocols may or may not be different. Thus, with a computing device 325 and a carrier board 375 in the remote device 300, the wireless connectivity and the RF components of the remote device 300 can be functionally separated. In some implementations, an antenna 382 is coupled to the RF unit 378 so that the RF unit 378 is configured to wirelessly communicate with the implant device 200.

In some implementations, one or more of the clock 320, the control system 330, the memory 340, the communications component 350, and any other electronic components of the remote device 300 may be powered by the power supply 360. The power supply 360 may be a battery, a solar cell, electrical socket, and other suitable power sources for harvesting power. The power supply 360 may also provide power to components of the carrier board 375.

In some implementations, the remote device 300 optionally includes a memory 372, a controller 374, a charger 376, and an RF unit 378 as part of the carrier board 375. The memory 372, which can include volatile memory, non-volatile memory (e.g., flash memory), or a combination thereof, can provide instructions to the controller 374. The controller 374, which can be used interchangeably with a “control system,” a “processor,” a “processing unit,” a microcontroller,” or a “control unit,” can be coupled to the memory 372 and control the operations of the charger 376 and the RF unit 378. The controller 374 may be in communication with components of the carrier board 375 and control operations of the carrier board 375. The controller 374 may include at least one of a general purpose single- or multi-chip processor, a DSP, an ASIC, an FPGA or other programmable logic device, discrete gate or transistor logic, or discrete hardware components.

The charger 376 can be configured to radiate a wireless signal from the remote device 300 to wirelessly charge a battery of another device, such as the implant device 200. In some implementations, the charger 376 can radiate a wireless signal in the near-field range or mid-field range. In some implementations, when the implant device 200 receives the wireless signal from the charger 376, the implant device 200 can be automatically configured to perform data transfer with the remote device 300.

The RF unit 378 can include one or more of a receiver, transmitter, and two-way transceiver to wirelessly communicate with another device, such as the implant device 200. In some implementations, the RF unit 378 in the carrier board 375 may be configured to transmit programming instructions for configuring the implant device 200, and may be configured to receive data (e.g., compliance data) from the implant device 200. In some implementations, the RF unit 378 may be configured to communicate with the implant device 200 in MICS band, MEDS band, or any other suitable frequency band using the antenna 382. The MICS band is an ultra-low power, unlicensed, mobile radio service for transmitting data in support of diagnostic or therapeutic functions associated with implanted medical devices. In some implementations, the RF unit 378 of the carrier board 375 is configured to wirelessly communicate with the implant device 200 while the communications component 350 of the computing device 325 is configured to communicate with a wireless communication hub, database system, or cellular device, such as a smart phone, mobile phone, smart watch, tablet, PDA, laptop computer, desktop computer, or other device with cellular communication capability, over a wired or wireless interface. In some implementations, one or more of the memory 372, the controller 374, the charger 376, and the RF unit 378 may be powered by the power supply 360.

The carrier board 375 may be configured to wirelessly and subcutaneously communicate with the implant device 200 and to wirelessly charge the implant device 200, and the computing device 325 may be configured to communicate data received from the implant device 200 to other devices (e.g., non-implanted devices). Thus, the carrier board 375 and the computing device 325 may be separate boards running different software. The wireless connectivity and the radio-frequency components of the remote device 300 can be “sandboxed” (i.e., functionally separated) with the computing device 325 so that such components are isolated from the carrier board 375. For example, the remote device 300 sandboxes or otherwise isolates the implant connectivity from cloud connectivity. Functions, programs, and/or components in the computing device 325 can be separated from functions, programs, and/or components in the carrier board 375. Separating the carrier board 375 from the computing device 325 in such a manner can separate the functionality of communicating with the implant device 200, which can involve FDA Class III functionality, from the functionality of communicating with other devices, which can involve FDA Class I/II functionality. Separating out the functionality in this manner can separate the processes so that communication with the implant device 200 is running FDA Class III software and so that communication with a wireless communication hub device, database system, or cellular device is running FDA Class I/II software. This can reduce the amount of testing that may be required for FDA approval of the remote device 300. Nonetheless, while the computing device 325 and the carrier board 375 may be separate boards running different software, the computing device 325 and the carrier board 375 may be integrated in a single device or apparatus, such as the remote device 300.

In some implementations, the remote device 300 can perform functions in addition to charging the implant device 200 and communicating with the implant device 200. Specifically, the remote device 300 can be a multifunctional device, such as a multifunctional wearable device. An example of a multifunctional wearable device can be a multifunctional watch or smart watch. In other examples, the multifunctional wearable device may be smart clothing, smart shoes, smart glasses, AR/VR headsets, smart gear, etc. In another example, the multifunctional device may be a smartphone, smart furniture, drones, automobiles, etc. In some implementations, the remote device 300 may also serve as a biometric monitoring device that tracks, reports, and/or communicates various biometric measurements, such as distance travelled, steps taken, flights of stairs climbed, calories burned, heart rate, hours of sleep, etc. In some implementations, the remote device 300 may also serve as a mobile phone or may be linked to a mobile phone to place and answer phone calls, send and receive text messages, and initiate voice commands. In some implementations, the remote device 300 may have audio functions, which can enable the remote device 300 to convey information through audio tones, voice, songs, or other sounds. In some implementations, the remote device 300 may include a display or touchscreen. The remote device 300 may display various types of information, such as time, date, and weather. Additionally, the remote device 300 may be able to access the internet and/or other devices, and may be able to pull data from the internet and/or other devices. Furthermore, the remote device 300 may be capable of displaying news, social network updates, email, reminders, calendar notifications, etc. In some implementations, the remote device 300 may include or be programmed with one or more “apps” that can provide additional functionality to the remote device 300. In some implementations, the remote device 300 may include accelerometers, gyroscopes, magnetometers, and/or other sensors, where the remote device 300 may collect, send, store, display, or analyze data from the sensors. The aforementioned concepts and functions of the remote device 300 are intended to be illustrative and it is to be understood that other concepts and functions can be part of the remote device 300 without departing from the scope of the present disclosure. As such, the scope of the disclosure is not limited to the description of the above concepts and functions.

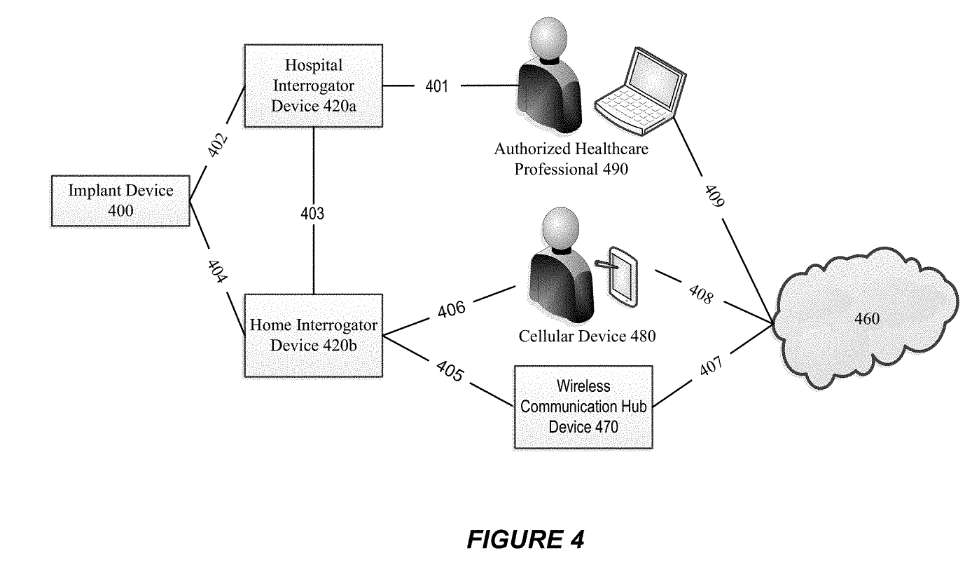

FIG. 4 shows a system diagram illustrating communication pathways in an example environment including an implant device, a “hospital” interrogator device, a “home” interrogator device, and a cloud-based database system according to some implementations. Implant devices, fitness devices, and electronic medical devices have been developed by a large number of manufacturers who have focused on the medical aspect of their products, but have only recently considered or added wireless communication capabilities. Such devices may be integrated in a system architecture that provides for secure communications therebetween. In some implementations, secure communications can be made from an implant device ultimately to a cloud-based database system, and vice versa.

As used herein, a “hospital” interrogator device is any remote device or remote electronic device configured to provide instructions for configuring one or more operations of an implant device and identification data to the implant device. A “home” interrogator device is any remote device or remote electronic device configured to pair with the implant device and receive data from the implant device. The hospital interrogator device and the home interrogator device may be used in different places and settings. Generally, the hospital interrogator device is designed to communicate with an implant device in a medical office setting such as a hospital environment, or when in possession with a healthcare professional. The home interrogator device is designed to communicate with the implant device in any environment, such as a patient's home or office, or outside of the possession of the healthcare professional. Nonetheless, it will be understood that the hospital interrogator device and the home interrogator device may be used in any environment, where the hospital interrogator device may be referred to as a first external interrogator device and the home interrogator device may be referred to as a second external interrogator device, or vice versa. Each of the hospital interrogator device and the home interrogator device can include components identical or similar to the components of the remote device shown in FIGS. 1 and 3. However, it will be understood that the components of the hospital interrogator device may include different, fewer, or additional components than the home interrogator device. As will be discussed in more detail below, the hospital interrogator device performs different functions than the home interrogator device in communicating with the implant device.

The environment in FIG. 4 includes an implant device 400 that can be implanted in a patient's body. The implant device 400 can include components identical or similar to the components of the implant device shown in FIGS. 1 and 2. The environment further includes a hospital interrogator device 420a and a home interrogator device 420b, both of which are external to the implant device 400. In some implementations, devices “external” to the implant device 400 refers to devices that are outside of the patient's body in which the implant device 400 is implanted. A doctor, healthcare professional, or other authorized user 490 may communicate directly with the hospital interrogator device 420a across a communication pathway 401. In some implementations, a user 490 directly interfaces with the hospital interrogator device 420a. In some implementations, a user 490 directly interfaces with the hospital interrogator device 420 via a display interface connected to the hospital interrogator device, such as a touchscreen. In some implementations, a user 490 interfaces with the hospital interrogator device 420a through a wired or wireless network from a cellular device, such as a smart phone, mobile phone, smart watch, tablet, PDA, laptop computer, desktop computer, or other device with cellular communication capability.

The hospital interrogator device 420a may be provisioned with credentials for authenticating one or more users to access the hospital interrogator device 420a. In some implementations, a user may input credentials for authentication using a login and password, using biometric data (e.g., fingerprint), or using a token-based authentication mechanism (e.g., SecurID). In communication pathway 401, the hospital interrogator device 420a may receive a request from a user to access the hospital interrogator device where the request includes authentication credentials, determine that the authentication credentials are valid, and establish secure communication between the implant device 400 and the hospital interrogator device 420a in communication pathway 402 after determining that the authentication credentials are valid. In some implementations, a server managed by a third-party broker may determine that the authentication credentials are valid. Depending on the authentication credentials provided by the user, some users will have access to certain features of the hospital interrogator device 420a depending on what privileges are assigned to each user. Authentication credentials associated with different users may have different privileges and access rights. Accordingly, some users may have access to some features of the hospital interrogator device 420a whereas other features are disabled, and some users may have access to all features of the hospital interrogator device 420a. Such features of the hospital interrogator device 420a may be accessible (or disabled) on a display or other interface on the hospital interrogator device 420a.

When authenticating a user for authorizing access to the hospital interrogator device 420a, a secure communication may be established between the hospital interrogator device 420a and the implant device 400 in communication pathway 402. A handshake protocol may be performed between the hospital interrogator device 420a and the implant device 400. To ensure integrity protection and confidentiality of the communication pathway 402, integrity and confidentiality keys may be generated using a secret key to perform authentication and key agreement. In some implementations, encryption and authentication keys may be generated by a server using the secret key. The secret key may be pre-provisioned both in the implant device 400 and the hospital interrogator device 420a (e.g., during the manufacturing of the device). In alternate implementations, authentication and key agreement may be performed using public/private key pairs of the implant device 400 and the hospital interrogator device 420a. The public/private key pairs can be either generated or pre-provisioned in each device independently using out-of-band mechanisms prior to the establishment of secure communication between the hospital interrogator device 420a and the implant device 400. Such secret keys or public/private key pairs may be used, for example, to securely add or remove interrogator devices, add or revoke privileges of interrogator devices, and authenticate code/patches downloaded to the implant device 400. The addition of an interrogator device 420a may be performed using any secure pairing method known in the art. In some implementations, private and/or public keys provisioned into the implant device 400 and the interrogator device 420a as part of the device manufacturing may be used to secure the aforementioned operations. Information transmitted across the communication pathway 402 may be encrypted (e.g., via AES 128) and integrity protected via a message authentication code using keys generated during the authentication and key agreement process. Information may be transmitted securely while maintaining the appropriate security and privacy required under government regulations (e.g., HIPAA).

When secure communication is established in communication pathway 402, the hospital interrogator device 420a may be configured to program or otherwise configure the operations of the implant device 400. In some implementations, the hospital interrogator device 420a may adjust the therapy or change the treatment profile of the implant device 400. For example, where the implant device 400 is a neural stimulator, the hospital interrogator device 420a may control the timing and amount of electrical signals being sent to the nerve. In some implementations, communications with the implant device 400 across the communication pathway 402 are limited to short-range (i.e., local) communications, such as the Bluetooth frequency band or MICS frequency band. The communication pathway 402 can subcutaneously reach within a patient's body, such as at least 12 cm within the patient's body. Accordingly, programming or otherwise configuring the implant device 400 can require the hospital interrogator device 420a to be in close proximity to the patient. In some implementations, communications with the implant device 400 across communication pathway 402 occurs via a local area network or direct physical connection. For example, programming or otherwise configuring the implant device 400 can require the hospital interrogator device 420a to be connected to a hospital network or to be directly connected with the implant device 400. The communication pathway 402 not only limits access to features of the hospital interrogator device 420a to authenticated users (e.g., authorized healthcare professionals), but the communication pathway 402 also limits remote access to the implant device 400.

The home interrogator device 420b may be configured to pair with the implant device 400 using the hospital interrogator device 420a. A home interrogator device 420b may have identity and/or credentials associated with it. The hospital interrogator device 420a may provide identification data to the implant device 400 for identifying the home interrogator device 420b. In some implementations, the hospital interrogator device 420a programs secret keys or public/private key pairs to the home interrogator device 420b across communication pathway 403 and programs secret keys or public/private key pairs to the implant device 400 across communication pathway 402. The secret keys or public/private key pairs may include the identity and/or credentials of the implant device 400 as well as the identity and/or credentials of the home interrogator device 420b. Rather than pre-provisioning each of the home interrogator device 420b and the implant device 400 with keys during manufacturing, secret keys or public/private key pairs may be generated via the hospital interrogator device 420a for pairing the implant device 400 with the home interrogator device 420b. Generation of such keys for securely pairing the implant device 400 with the home interrogator device 420b is not necessarily dependent on a third-party system or third-party server. When the implant device 400 identifies the home interrogator device 420b, the implant device 400 may be paired with the home interrogator device 420b. Upon pairing, the home interrogator device 420b is configured to receive data, such as physiological data, compliance data, or other data collected/generated from the implant device 400, across communication pathway 404. Communication of such data across the communication pathway 404 may be limited to short-range (i.e., local) communications, such as Bluetooth frequency band or the MICS frequency band. In some implementations, the pairing the home interrogator device 420b and the implant device 400 does not occur unless the home interrogator device 420b is authenticated by the implant device 400.

In some implementations, the home interrogator device 420b may be provisioned with credentials for authenticating one or more users to access the home interrogator device 420b. When authenticating a user for authorizing access to the home interrogator device 420b, a secure communication may be established between the home interrogator device 420b and the implant device 400 in communication pathway 404. The implant device 400 establishes session keys with the home interrogator device 420b using an authenticated key agreement protocol. Data transmission across the communication pathway 404 can be integrity protected and encrypted using the session keys.

To ensure integrity protection and confidentiality of the communication pathway 404, integrity and confidentiality keys may be generated using a secret key to perform authentication and key agreement. In some implementations, encryption and authentication keys may be generated by using the secret key during the authentication and key agreement. In alternate implementations, authentication and key agreement may be performed using public/private key pairs. The secret key or the public/private key pairs may be agreed between the implant device 400 and the home interrogator device 420b as part of the pairing using the hospital interrogator device 420a. The secret key or the public/private key pairs may be programmed by the hospital interrogator device 420a to the implant device 400 via communication pathway 402 and by the hospital interrogator device 420a to the home interrogator device 420b via communication pathway 403. Such secret keys or public/private key pairs may be used, for example, to securely add or remove interrogator devices, add or revoke privileges of interrogator devices, and authenticate code/patches downloaded to the implant device 400. The addition of an interrogator device 420b may be performed using any secure pairing method known in the art. In some implementations, private and/or public keys provisioned into the implant device 400 and the interrogator device 420b as part of the device manufacturing may be used to secure the aforementioned operations. Information transmitted across the communication pathway 404 may be encrypted (e.g., via AES 128) and integrity protected via a message authentication code using keys generated during the authentication and key agreement process. Information may be transmitted securely while maintaining the appropriate security and privacy required under government regulations (e.g., HIPAA).

The home interrogator device 420b may be configured to receive data from the implant device 400 when securely paired, and may be configured to upload such data to other electronic devices in the environment in FIG. 4. In some implementations, the home interrogator device 420b may be configured to upload such data to a cloud-based database system 460. However, it will be understood that the data may be uploaded to any server and is not necessarily limited to a cloud-based database system 460. The cloud-based database system 460 may include a server in the cloud configured to store data collected from the implant device 400. In FIG. 4, the home interrogator device 420b receives data from the implant device 400 across communication pathway 404, and uploads the data from the home interrogator device 420b to the cloud-based database system 460 across communication pathways 405-408. However, the home interrogator device 420b may be programmed or otherwise configured to only receive data from the implant device 400 and not program or configure the implant device 400 itself. Accordingly, in some implementations, the home interrogator device 420b may be unable to program or reprogram operations of the implant device 400, thereby limiting the extent to which the home interrogator device 420b can autonomously change therapy or change a treatment profile.

The functions, capabilities, and configurations of the hospital interrogator device 420a, the home interrogator device 420b, and the implant device 400 can ensure multiple levels of security and data protection. The ability to configure or program the implant device 400 may be limited to being close to the patient. In some instances, the ability to configure or program the implant device 400 may be limited to being within a hospital network or in direct physical connection with the implant device 400. Additionally, the ability to configure or program the implant device 400 may be limited to those in possession of the specific hospital interrogator device 420a with the appropriate identity and credentials. In other words, the ability to configure or program the implant device 400 may be limited to those who have been authenticated by the hospital interrogator device 420a with the appropriate authentication credentials. That way, only authorized doctors, authorized healthcare professionals, or authorized users 490 may access the implant device 400 through the hospital interrogator device 420a. In some implementations, pairing between the home interrogator device 420b and the implant device 400 across communication pathway 404 may not occur without generation of secret keys or public/private key pairs from the hospital interrogator device 420a. Moreover, the home interrogator device 420b may be configured to only receive data from the implant device 400, or at least not be able to autonomously configure or program the implant device 400.

To relay data from the home interrogator device 420b to the cloud, the home interrogator device 420b may securely pair with other electronic devices external to the implant device 400, such as a wireless communication hub device 470 or a cellular device 480. The home interrogator device 420b may be capable of communicating with these electronic devices external to the implant device 400 via a wide area network, personal area network, local area network, and near-field communication (NFC). The home interrogator device 420b may communicate with the wireless communication hub device 470, such as a 2Net hub or similar device, across a communication pathway 405. In some implementations, the communication pathway 405 can correspond to a Bluetooth or Wi-Fi communication protocol. The home interrogator device 420b may communicate with the cellular device 480, such as a smart phone, mobile phone, smart watch, tablet, PDA, laptop computer, desktop computer, or other device with cellular communication capability, across a communication pathway 406. In some implementations, the communication pathway 406 can correspond to a Bluetooth or Wi-Fi communication protocol. As shown in FIG. 4, the implant device 400 does not directly pair with a cellular device 480, but pairs with a home interrogator device 420b, that in turn pairs with the cellular device 480 or the wireless communication hub device 470. The home interrogator device 420b may have the link budget to reach the required depth inside a patient's body and wirelessly communicate with the implant device 400 that a conventional cellular device 480 may not possess. In some implementations, the home interrogator device 420b may be a wearable device. Examples of a wearable device can include a watch, collar, belt, or other wearable. In some implementations, the home interrogator device 420b may be placed in close proximity to the patient. For example, the home interrogator device 420b may be placed on a patient's bed or bedsheet while the patient is sleeping.

The wireless communication hub device 470 may be configured to be pre-paired with the home interrogator device 420b, where the wireless communication hub device 470 is configured to readily recognize the identity and credentials of the home interrogator device 420b and the home interrogator device 420b is configured to readily recognize the identity and credentials of the wireless communication hub device 470. In other words, the wireless communication hub device 470 can be “pre-provisioned” to work with the home interrogator device 420b and the home interrogator device 420b can be “pre-provisioned” to work with the wireless communication hub device 470. In some implementations, the wireless communication hub device 470 can select the home interrogator device 420b from a white list of allowed devices and avoid connecting with devices on a black list of prohibited devices. That way, the wireless communication hub device 470 can quickly establish a network interface from the implant device 400 to the cloud-based database system 460.

The wireless communication hub device 470 or the cellular device 480 may receive the data from the home interrogator device 420b and upload such data to the cloud-based database system 460. The wireless communication hub device 470 can securely upload the data to the cloud-based database system 460 across a communication pathway 407. In some implementations, the communication pathway 407 can correspond to a WWAN (e.g., 3G cellular wireless network) communication protocol. The cellular device 480 can securely upload the data to the cloud-based database system 460 across a communication pathway 408. In some implementations, the communication pathway 408 can correspond to a WWAN (e.g., 3G or 4G cellular wireless network) communication protocol.

The wireless communication hub device 470 may ensure integrity protection and encryption of data for secure end-to-end communication from the home interrogator device 420b to the cloud-based database system 460. An authorized user may be authenticated to access the data from the cellular device 480 if it is determined that his/her authentication credentials are valid. If authenticated, then the communication pathway 406 between the home interrogator device 420b and the cellular device 480 may be established. Secret keys generated by a server managed by a third-party broker may be transmitted to the cellular device 480 and used to access the home interrogator device 420b. Establishment of the communication pathway 406 may require authentication and key agreement to be performed using the secret key. In some implementations, session keys established using the authentication and key agreement process may be used to provide data integrity and encryption.

The data from the implant device 400 may be stored in the cloud-based database system 460, and may be accessible by an authorized user 490, such as an authorized doctor or healthcare professional. The authorized user 490 may access the stored data in the cloud-based database system 460 across a communication pathway 409. The authorized user 490 may be authenticated to access the data stored in the cloud-based database system 460 if his/her authentication credentials are valid. In some implementations, the authorized user 490 may access the data stored in the cloud-based database system 460 using his/her cellular device. The authorized user 490 need not be in close proximity to the patient to access such data. The authorized user 490 is capable of changing the therapy of the implant device 400 in response to viewing the data accessed from the cloud-based database system 460. For example, a doctor can access the data provided from the implant device 400 and, depending on the results, the doctor can adjust the therapy and program the implant device 400 appropriately. However, in some implementations, the implant device 400 can be programmed to adjust therapy when the data satisfies one or more predetermined conditions without intervention from the doctor.

In some implementations, in the event that either the hospital interrogator device 420a or the home interrogator device 420b fails (e.g., chip dies) or otherwise needs replacement, a replacement interrogator device 420a, 420b may be provisioned. For example, private and/or public keys may be provisioned into the interrogator device 420a, 420b as part of the device manufacturing. In some implementations, encryption and authentication keys may be generated by a server managed by a third-party broker using a secret key to program a replacement interrogator device 420a, 420b. In some implementations, private and/or public keys may be generated using the hospital interrogator device 420a when replacing the home interrogator device 420b.

In FIG. 4 according to some implementations, data transmitted from the implant device 400 to the interrogator devices 420a, 420b and vice versa may occur over a short-range communication protocol (e.g., MICS communication protocol or Bluetooth communication protocol). In some implementations, data transmitted from the home interrogator device 420b to the wireless communication hub device 470 or the cellular device 480 may occur over a local area network (e.g., Wi-Fi) or personal area network (e.g., Bluetooth) communication protocol. In some implementations, data transmitted from the wireless communication hub device 470 or the cellular device 480 to a platform server (e.g., 2Net platform server) or cloud-based database system 460 may occur over a wide area network (e.g., WWAN) communication protocol. The environment shown in FIG. 4 illustrates a system architecture that provides end-to-end connectivity from the implant device 400 to devices external to the implant device 400.

FIG. 5 shows a flow diagram illustrating an example method for establishing secure communication between an implant device and an external home interrogator device according to some implementations. A method 500 may be performed in a different order or with different, fewer, or additional operations. In some implementations, the blocks of the process 500 may be performed by a control system of an external hospital interrogator device, such as an external hospital interrogator device 300 shown in FIG. 3. Although some blocks are of the method 500 may be described as being performed by a single processor of the external hospital interrogator device, in alternative implementations, more than one processor may be involved in performing the operations.

At block 505 of the method 500, the external hospital interrogator device authenticates the user, where the external hospital interrogator device 500 is provisioned with user credentials for authenticating one or more users to access the external hospital interrogator device. In some implementations, the user credentials provisioned in the external hospital interrogator device may include a login and password, biometric data, or a token-based authentication mechanism.

Upon successful user authentication, a user is determined to have privileges to access features for configuring one or more operations of the implant device. Such users may be limited to certain authorized doctors or healthcare professionals. The user credentials provisioned in the external hospital interrogator device may be associated with different privileges and access rights. Depending on the user credentials provided, the user may have certain privileges in accessing features of the external hospital interrogator device. The user may be authorized to access some features for configuring the operations of the external hospital interrogator device whereas some features may be disabled.

At block 510, communication is established between the external hospital interrogator device and the implant device. In some implementations, secret keys or public/private key pairs are provisioned in the external hospital interrogator device and the implant device. A secure communication may be established between the external hospital interrogator device and the implant device using the pre-provisioned secret keys or public/private key pairs. In some implementations, the secret keys or public/private key pairs are provisioned as part of the device manufacturing processes. A handshake protocol may be performed between the external hospital interrogator device and the implant device to establish secure communication.

At block 515 of the method 500, one or more operations of the implant device is configured using the external hospital interrogator device. The one or more operations of the implant device may be controlled by a control system of the implant device. In some implementations, the one or more operations include changing a therapy or treatment profile of the implant device. For example, where the implant device is a neural stimulator, changing a therapy or treatment profile can include changing a timing or amount of electrical pulses sent to a nerve. In some implementations, the one or more operations include wirelessly charging a battery of the implant device. In some implementations, the one or more operations include adjusting a polling rate of one or more sensors of the implant device. In some implementations, the one or more operations include changing a wake-up protocol of the implant device. Accordingly, an authorized user may have privileges and/or access rights to controlling some or all of the aforementioned operations of the implant device.

At block 520 of the method 500, identification data is provided to the implant device and the external home interrogator device for pairing the implant device to an external home interrogator device. The identification data may include identity and/or credentials for identifying and authenticating the external home interrogator device and the implant device for establishing secure communication between the devices. The identification data may be provided by the external hospital interrogator device after the user is authenticated and the secure communication is established between the external hospital interrogator and the implant device. In some implementations, the identification data includes a secret key or public/private key pairs provided by the user-authenticated external hospital interrogator device. The secret key or public/private key pairs are not necessarily provided as part of the device manufacturing processes. That way, pairing between the implant device and the external home interrogator device does not occur apart from the identification data provided by the external hospital interrogator device, where the external hospital interrogator device is accessible to authorized users, such as doctors and healthcare professionals. It will be understood that the operation of block 520 may occur after the operation of block 515, the operation of block 520 may occur before the operation of block 515, or the operation of block 520 and the operation of block 515 may occur simultaneously.

FIG. 6 shows a flow diagram illustrating an example method for transmitting physiological data from an implant device to an external home interrogator device according to some implementations. A method 600 may be performed in a different order or with different, fewer, or additional operations. In some implementations, the blocks of the process 600 may be performed by a control system of an implant device, such as an implant device 200 shown in FIG. 2. Although some blocks are of the method 600 may be described as being performed by a single processor of the external hospital interrogator device, in alternative implementations, more than one processor may be involved in performing the operations.

At block 605 of the method 600, instructions are received from an external hospital interrogator device for configuring the implant device according to a treatment profile. Before the implant device can be paired with another electronic device to communicate the physiological data, and before the implant device can be configured, an external hospital interrogator device is authenticated and secure communication is established with the implant device. The external hospital interrogator device may be authenticated according to user credentials and protocols discussed above. Authentication limits configuring the implant device and limits providing identification data for pairing the implant device to authorized users of the external hospital interrogator device.

At block 610 of the method 600, identification data is received from the external hospital interrogator device for pairing the implant device with an external home interrogator device. The identification data may include one or both of an identity and credentials of the external home interrogator device. For example, the identification data can include a secret key or public/private key pairs provided by the external hospital interrogator device. An implant device may be programmed with an identity for identifying the external home interrogator device and with credentials for authenticating one or more users to access the external home interrogator device. The external home interrogator device may be configured to receive data from the implant device when paired with the implant device, but is configured to not program or otherwise configure operations of the implant device. Such features may be disabled in the external home interrogator device.

At block 615 of the method 600, the external home interrogator device is identified to pair the implant device with the external home interrogator device. Using the identification data to identify the external home interrogator device when the external home interrogator device is within close proximity to the implant device, pairing between the external home interrogator device and the implant device can be established. The pairing mechanism can be any pairing mechanisms known in the art that results in a secret key being agreed between the implant device and the external home interrogator device. The pairing between the external home interrogator device and the implant device may occur over a short-range communication protocol, such as a MICS communication protocol or a Bluetooth communication protocol. In some implementations, identification of external home interrogator device for pairing occurs upon receipt of a triggering signal. The triggering signal can initiate pairing between the implant device and the external home interrogator device. In some implementations, the triggering signal is a charging signal for charging the implant device or other signal indicating close proximity to the implant device.

At block 620 of the method 600, physiological data is obtained from one or more sensors of an implant device based on the treatment profile. The implant device may be equipped with one or more sensors to measure and/or collect physiological data regarding a patient. The one or more sensors can include but is not limited to temperature sensors, chemical sensors (e.g., blood glucose sensors), pressure sensors, pulse sensors, piezoelectric sensors, electric field sensors, electromyographic sensors, respiration sensors, moisture sensors, optical sensors, and other biomedical sensors. The physiological data may include physiological signals, biometric information, conditions, or parameters associated with the patient as treatment is being delivered to the patient. Examples of physiological data can include but is not limited to blood pressure, heart rate, pulse rate, EEG, EKG, ECG, skin conduction, body or skin temperature, weight, body fat, respiration rate, blood flow, oxygen level, CO2 level, and glucose level, among others. It will be understood that the operations of block 620 may occur at any time after the operation of block 605 and before the operation of block 625.

At block 625 of the method 600, the physiological data is transmitted to the paired external home interrogator device. In some implementations, session keys are established from the secret key using an authenticated key agreement protocol for transmitting the physiological data to the external home interrogator device. The session keys established from the authenticated key agreement protocol allows for integrity protection, confidentiality, and security of messages transmitted between the implant device and the external home interrogator device. In some implementations, the paired external home interrogator device is a wearable device, such as a multifunctional wearable device. In some implementations, the physiological data may be uploaded to a cloud-based database system via a wireless communication hub device or a cellular device. As such, the physiological data may then be accessible to a patient and/or authorized healthcare professional.

FIG. 7A shows a flow diagram illustrating an example method of operating an implant device according to some implementations. The process 700a may be performed in a different order or with different, fewer, or additional operations. In some implementations, the blocks of the process 700a may be performed by the implant device 200 shown in FIGS. 1, 2, and 4. Although some blocks are of the method 700a are described as being performed by a single processor of the implant device, in alternative implementations, more than one processor may be involved in performing the operations. For example, the blocks of the method 700a may be performed by a control system in the implant device.

At block 705 of the method 700a, physiological data associated with a treatment profile of a patient is received via one or more sensors in the implant device. The one or more sensors can include but are not limited to temperature sensors, chemical sensors (e.g., blood glucose sensors), pressure sensors, pulse sensors, piezoelectric sensors, electric field sensors, electromyographic sensors, respiration sensors, moisture sensors, optical sensors, and other biomedical sensors. The physiological data may include physiological signals, biometric information, conditions, or parameters associated with the patient as treatment is being delivered to the patient. Examples of physiological data can include but is not limited to blood pressure, heart rate, pulse rate, EEG, EKG, ECG, skin conduction, body or skin temperature, weight, body fat, respiration rate, blood flow, oxygen level, CO2 level, and glucose level, among others. The physiological data of the patient may be collected and analyzed by the implant device. In some implementations, the physiological data may be calculated, estimated, processed, and/or analyzed by one or more processors of the implant device. In some implementations, the raw physiological data or the analyzed physiological data may be stored in the memory of the implant device.

The physiological data received from the implant device may be indicative of one or more conditions of the patient. The physiological data may be analyzed to determine a health-related condition or provide diagnostic information of the patient. By way of some examples, the physiological data can be utilized to ascertain whether the patient is having an asthma attack, whether the patient is sleeping or not, whether the patient is having a heart attack or not, whether the patient is having a migraine headache or not, whether the patient is having a seizure or not, etc. In some implementations, a treatment profile or therapy of the implant device may change in response to the physiological data received from the one or more sensors. The implant device may be configured to automatically change a treatment profile or therapy in response to one or more conditions of the patient being satisfied.

Physiological data collected and gathered from the implant device inside the patient may not be sufficient to configure the treatment profile or therapy of the implant device. Non-physiological data may be collected and gathered to supplement the physiological data so that more information regarding the patient can be provided. The implant device may be integrated in a system architecture that allows for secure communications not only with interrogator medical devices, but with wireless communication hubs, cellular devices, cloud-based database systems, and the like. Such devices that are external to the implant device inside the patient may be readily accessible to the implant device. This can occur when the implant device is securely paired with an interrogator device, regardless of whether the patient is in the hospital or not. Such devices can provide additional information regarding the patient and the environment that the patient is in.

At block 710 of the process 700a, non-physiological data from the implant device or from a device external to the implant device is retrieved. Such non-physiological data can include, for example, environmental data (e.g., air quality, temperature, pressure, time of day, GPS or position data, humidity, etc.), activity data (e.g., running, sleeping, walking, bicycling, swimming, standing, climbing stairs, etc.), user data (e.g., height, weight, body fat, known health conditions, medication intake, etc.), system condition data (e.g., low battery, low memory, etc.), and contextual data (e.g., weather patterns at a certain time or year, health risks in certain geographical locations, etc.). In some implementations, non-physiological data may be retrieved from an interrogator device that is paired with the implant device, or from one or more components of the implant device itself. The interrogator device may be configured to wirelessly retrieve data from various sources, such as a wireless communication hub, cellular device, or cloud-based database system. The interrogator device may be configured to wirelessly retrieve data from the internet. In addition or in the alternative, the interrogator device may include one or more environmental sensors, biometric sensors, or other sensors. Environmental sensors may detect, measure, and/or sense ambient environmental conditions, such as ambient pressure, temperature, sound, light, humidity, location, and atmosphere. For example, an environmental sensor can include an atmospheric sensor configured to acquire data representative of air quality, pollen count, carbon dioxide, carbon monoxide, and odor, among others. When the interrogator device is successfully paired with the implant device, the interrogator device may access and retrieve non-physiological data from various sources. In some implementations, the interrogator device is a wearable interrogator device.

At block 715 of the process 700a, the non-physiological data, alone or in combination with the physiological data, is determined to satisfy a condition. The determination may be made via one or more processors of the implant device. One or more conditions or parameters may be configured in the implant device, where the one or more conditions or parameters are compared against the non-physiological data and/or physiological data. Satisfaction of the one or more conditions or parameters may serve as triggers for causing a response to be initiated in the implant device. In some implementations, the one or more conditions or parameters may be stored in the memory of the implant device. In some implementations, the one or more conditions or parameters may be represented by threshold values, where the threshold values may be user-defined or system-defined. The non-physiological and/or physiological data may be processed and analyzed prior to determining whether a condition is satisfied or not.

At block 720 of the process 700a, an operation of the implant device is adjusted in response to determining that the condition has been satisfied. Satisfaction of the condition may trigger or otherwise cause the implant device to deliver appropriate therapy or treatment in response. As discussed earlier, the implant device may include but is not limited to cardiac pacemakers, ICDs, PCDs, brain stimulators, gastric system stimulators, nerve or neural stimulators, muscle stimulators, lower colon stimulators, drug dispensers or pumps, cardiac signal loops or other types of recorders or monitors, gene therapy delivery devices, prevention or monitoring devices, insulin pumps or monitoring devices, and so on. In some implementations, the implant device is a neural stimulator that may control the stimulation pulses for delivery to tissue of the patient. The stimulation pulses may be increased or decreased depending on what conditions are met by non-physiological and/or physiological data.

The treatment profile of an implant device may be adjusted depending on physiological data of the patient. By way of an example, an insulin pump can deliver insulin depending on the detected blood glucose levels of the patient. A brain stimulator can send electrical impulses to parts of the brain depending on the neural activity (e.g., EEG) of the brain.

The treatment profile of an implant device may be adjusted not only based on physiological data but also from non-physiological data. By way of an example, one or more environmental sensors in an interrogator medical device can gather environmental data, such as temperature, humidity, CO2 levels, ambient pressure, air quality, level of allergens, types of allergens, and more. When one or more allergens are detected beyond a threshold level, antihistamines may be delivered by the implant device in response to the detected allergens.

Not only may a treatment profile or therapy of an implant device be adjusted based on physiological data and/or non-physiological data, but any adjustments to the operation of the implant device may be made. In some implementations, a polling rate of the one or more sensors of the implant device may be adjusted in response to determining that a condition has been satisfied. By way of an example, if one or more environmental sensors detect a high level of allergens, then the polling rate of the implant device may be increased. Specifically, the polling rate of the implant device may be increased from every 30, 40, 50, or 60 seconds to every 20, 15, 10, or 5 seconds. A high level of allergens may be indicative of a high chance of an asthma attack. Electrical stimulation from the implant device can alleviate an asthma attack. By way of another example, if one or more biometric sensors detect that the patient is jogging or running, then the polling rate of the implant device may be increased. By way of another example, if the weather pattern for a particular time of year indicates a higher likelihood of a high level of allergens, then the polling rate of the implant device may be increased. By way of another example, if a humidity sensor detects that a high level of moisture in the air and the patient is known to have arthritis, then the polling rate of the implant device may be increased. Electrical stimulation from the implant device may alleviate symptoms of arthritis. Thus, the operations of the implant device may be configured to change if non-physiological information, either alone or in combination with the physiological information, satisfies one or more conditions.

FIG. 7B shows a flow diagram illustrating an example method of operating an implant device according to some other implementations. The process 700b may be performed in a different order or with different, fewer, or additional operations. In some implementations, the blocks of the process 700b may be performed by the implant device 200 shown in FIGS. 1, 2, and 4. Although some blocks are of the method 700b are described as being performed by a single processor of the implant device, in alternative implementations, more than one processor may be involved in performing the operations. For example, the blocks of the method 700b may be performed by a control system in the implant device.

At block 755 of the process 700b, physiological data associated with a treatment profile of a patient is received via one or more sensors of an implant device. The operations of block 755 of the process 700b may be identical or at least similar to the operations of block 705 of the process 700a.

At block 760 of the process 700b, the physiological data is used to determine whether a specified physiological condition is satisfied. One or more physiological conditions or parameters may be user-defined or system-defined in the implant device. Satisfaction of the one or more specified physiological conditions can serve as triggers for initiating certain treatment by the implant device. In some implementations, the specified physiological condition can represent the onset of an adverse health condition. The physiological data received by the one or more sensors may be processed by one or more processors of the implant device. The processed physiological data may be compared against one or more conditions or parameters in the implant device.

If the physiological data satisfies a specified physiological condition at block 760, then a therapy of the implant device is adjusted via one or more processors of the implant device at block 765. The adjustment in therapy can be made in response to detection of the onset of an adverse health condition. For example, if the specified physiological condition is indicative of the onset of asthma, then a therapy may be delivered to the nerve in the lung in which the implant device is attached to. Otherwise, the continued therapy of the implant device may deliver only a mild amount of stimulation to the nerve, whereas an adjustment in therapy may deliver an increased amount of stimulation to the nerve.

If the physiological data does not satisfy a specified physiological condition at block 760, then the present therapy of the implant device continues. Furthermore, environmental data from a device external to the implant device is received via an RF unit of the implant device at block 770. The implant device may be integrated in a system architecture that facilitates secure communications with devices external to the implant device. Such devices may be equipped with one or more environmental sensors for acquiring environmental data. Environmental data may include but is not limited to ambient pressure, temperature, sound, light, humidity, location, air quality, pollen count, carbon dioxide, and odor. In some implementations, an interrogator medical device, such as a wearable interrogator medical device, may retrieve environmental data regarding an environment of a patient with the implant device. The environmental data may be transmitted across a secure communication pathway to the implant device. In some implementations, the environmental data may be transmitted in the MICS band or a near-field communication band when the interrogator device is securely paired with the implant device.

At block 775, the environmental data is used to determine whether a specified environmental condition is satisfied. One or more environmental conditions or parameters may be user-defined or system-defined in the implant device. Satisfaction of the one or more specified environmental conditions can serve as triggers for initiating certain operations by the implant device. Such operations are not necessarily limited to changes in treatment or therapy of the implant device. In some implementations, the specified environmental condition can provide information to the implant device of health-related concerns in the environment around the patient. For example, the specified environmental condition can detect allergens that the patient is breathing in, potentially harmful weather patterns, high humidity levels, poor air quality, etc. The environmental data received from a device external to the implant device may be processed by one or more processors of the implant device. The processed environmental data may be compared against one or more conditions or parameters in the implant device.

If the physiological data satisfies a specified environmental condition at block 775, then a polling rate of the one or more sensors in the implant device is adjusted via one or more processors of the implant device at block 780. In some implementations, polling a sensor can include reading one or more signals from the sensor. Polling can be carried out cyclically, such as about every 5, 10, 15, 20, 30, 40, 50, 60, 90, or 120 seconds or every 3, 5, 10, 30, 45, 60, or 120 minutes. That way, polling occurs at certain time periods or at certain time intervals, which can reduce power consumption by the implant device. Depending on the specified environmental condition that is satisfied, the polling rate of the one or more sensors can increase or decrease. For example, detection of a high humidity level can cause the polling rate of the implant device to increase for a patient with rheumatoid arthritis. In another example, detection of certain allergens or a high level of allergens in the air can cause the polling rate of the implant device to increase for a patient with asthma.

If the environmental data does not satisfy a specified environmental condition at block 775, then the polling rate of the one or more sensors of the implant device remains unchanged. The physiological data and the environmental data may be stored in the memory of the implant device for future use. In addition, the operations of the implant device repeats at block 755, where physiological data associated with a treatment profile of the patient is received via the one or more sensors of the implant device. The implant device continues detection of conditions inside and outside the patient, determination of satisfaction of one or more physiological or environmental conditions, and adjusting the operations of the implant device in response to such determinations.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the various embodiments.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

The functions in the various embodiments may be implemented in hardware, software, firmware, or any combination thereof. If implemented in software, the functions may be stored as one or more instructions or code on a non-transitory computer-readable medium or non-transitory processor-readable medium. The operations of a method or algorithm disclosed herein may be embodied in a processor-executable software module that may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiments shown herein but is to be accorded the widest scope consistent with the following claims and the principles and novel features disclosed herein.

What is claimed is:

1. An implantable medical device, comprising:
a radio-frequency (RF) communications circuitry;
one or more sensors configured to measure and/or collect physiological data; and
a control system coupled to the RF communications circuitry and coupled to the one or more sensors, wherein the control system is configured to:
receive instructions from a first external interrogator device for configuring the implantable medical device when the first external interrogator device is authenticated; and
receive identification data from the first external interrogator device for pairing the implantable medical device with a second external interrogator device.

2. The implantable medical device of claim 1, wherein the identification data includes a secret key or public/private key pairs provided by the first external interrogator device.

3. The implantable medical device of claim 1, wherein the first external interrogator device is provisioned with user credentials for authenticating one or more users to access the first external interrogator device.

4. The implantable medical device of claim 1, wherein the control system is configured to cause the RF communications circuitry to transmit the physiological data to the second external interrogator device when the second external interrogator device is paired with the implantable medical device.

5. The implantable medical device of claim 3, wherein the RF communications circuitry is configured to cause the RF communications circuitry to transmit the physiological data to the second external interrogator device in a Medical Implant Communications Service (MICS) frequency band or a Bluetooth frequency band.

6. The implantable medical device of claim 1, wherein the second external interrogator device includes a carrier board and a computing device, wherein the carrier board includes an RF unit for wirelessly communicating with the implantable medical device and the computing device includes a wireless communications component for wirelessly communicating with a wireless communication hub or a cellular device.

7. The implantable medical device of claim 1, further comprising:
wireless charger; and
a rechargeable battery coupled to the wireless charger, wherein the wireless charger is configured to receive signals to wirelessly charge the rechargeable battery in a mid-field frequency range between about 100 MHz and about 5 GHz or in a near-field frequency range.

8. The implantable medical device of claim 7, further comprising:
a clock die; and
a power management integrated circuit (PMIC) die, wherein the clock die and the PMIC die are constructed at a fabrication node configured for direct attachment to the rechargeable battery without protection circuitry.

9. The implantable medical device of claim 7, wherein the wireless charger and the RF communications circuitry share an antenna configured to receive and/or transmit signals in the near-field frequency range.

10. The implantable medical device of claim 7, wherein the control system is further configured to:
receive a trigger signal from the wireless charger; and
pair the implantable medical device with the second external interrogator device upon receipt of the trigger signal.

11. The implantable medical device of claim 1, wherein the control system is further configured to:
pair the implantable medical device with the second external interrogator device after the second external interrogator device is authenticated; and
establish session keys using an authenticated key agreement protocol for transmitting the physiological data to the second external interrogator device.

12. The implantable medical device of claim 1, wherein the control system is configured to receive instructions from the first external interrogator device via a local area network or direct physical connection.

13. The implantable medical device of claim 1, wherein the one or more sensors are configured to measure electrical stimulation activity of a nerve.

14. An interrogator device, comprising:
a first wireless communications component configured to communicate subcutaneously with an implant device;
a second wireless communications component configured to communicate with an electronic device external to the implant device; and
a control system configured to:
receive identification data for pairing the interrogator device with the implant device;
identify the implant device using the identification data to pair the interrogator device with the implant device; and
receive physiological data from the implant device protected using session keys established using an authenticated key agreement protocol.

15. The interrogator device of claim 14, wherein the identification data includes a secret key or public/private key pairs provided to the implant device by a remote device.

16. The interrogator device of 15, wherein the remote device is provisioned with user credentials for authenticating one or more users to access the remote device.

17. The interrogator device of claim 14, further comprising:
a carrier board, wherein the carrier board includes the first wireless communications component; and
a computing device, wherein the computing device includes the second wireless communications component, wherein the carrier board and the computing device are in communication with each other via a bidirectional communication interface.

18. The interrogator device of claim 14, wherein the first wireless communications component is configured to communicate subcutaneously with the implant device in a MICS frequency band or in a Bluetooth frequency band, and wherein the second wireless communications component is configured to communicate with the electronic device over one or more of a wide area network, personal area network, local area network, near-field communication (NFC) or any combination thereof.

19. The interrogator device of claim 18, wherein the second wireless communications component is configured to communicate with the electronic device in a Bluetooth frequency band or a Wi-Fi frequency band.

20. The interrogator device of claim 14, wherein the electronic device is a cellular device or a wireless communications hub device, and wherein the electronic device is configured to transmit the physiological data to a cloud-based database system.

21. The interrogator device of claim 14, wherein the interrogator device is integrated in a multifunctional wearable device.

22. The interrogator device of claim 14, further including a wireless charger coupled to the first wireless communications component, wherein the wireless charger is configured to wirelessly charge the implant device.

23. An interrogator device, comprising:
a wireless communications component configured to communicate subcutaneously with an implant device;
a memory configured to store user credentials for authenticating one or more users to access the interrogator device; and
a control system configured to:
authenticate a user to access the interrogator device using the user credentials;
establish secure communication between the interrogator device and the implant device; and
transmit, via the wireless communications component, instructions to the implant device to configure one or more operations of the implant device.

24. The interrogator device of claim 23, wherein the control system is further configured to:
provide identification data to a remote device and the implant device for pairing the remote device and the implant device, wherein the identification data includes a secret key or public/private key pairs.

25. The interrogator device of claim 23, wherein the control system is configured to transmit instructions to the implant device via a local area network or direct physical connection.

26. The interrogator device of claim 23, wherein the control system is further configured to:
determine, after authenticating the user to access the interrogator device, that the user has privileges to be able to access features for transmitting instructions to the implant device.

27. The interrogator device of claim 23, wherein the wireless communications component is further configured to wirelessly charge a battery in the implant device.

28. A method of operating an implant device, comprising:
receiving, via one or more sensors of an implant device, physiological data associated with a treatment profile of a patient;
receiving non-physiological data from the implant device or from a device external to the implant device;
determining that the non-physiological data, alone or in combination with the physiological data, satisfies a condition; and
adjusting, via one or more processors of the implant device, an operation of the implant device in response to determining that the condition has been satisfied.

29. The method of claim 28, wherein the non-physiological data includes one or more of environmental data, activity data, user data, system condition data, and contextual data.

30. The method of claim 29, wherein the non-physiological data includes environmental data received from the device external to the implant device, the environmental data including one or more of ambient pressure, temperature, sound, light, humidity, location, air quality, pollen count, carbon dioxide, and odor.

31. The method of claim 28, wherein adjusting an operation of the implant device includes adjusting a polling rate of the one or more sensors of the implant device.

32. The method of claim 28, wherein adjusting an operation of the implant device includes adjusting the treatment profile of the patient.

33. The method of claim 28, wherein receiving the non-physiological data includes receiving non-physiological data from the device external to the implant device when the device external to the implant device is authenticated.
